(19) United States
(12) Patent Application Publication
(10) Pub. No.: US 2018/0192966 A1
(43) Pub. Date: Jul. 12, 2018

(54) IMAGE ACQUISITION DEVICE AND METHOD

(71) Applicants: Rayence Co., Ltd., Gyeonggi-do (KR); VATECH EWOO Holdings Co., Ltd., Gyeonggi-do (KR)

(72) Inventors: Tae Woo KIM, Gyeonggi-do (KR); In Jae BAEK, Gyeonggi-do (KR)

(73) Assignees: Rayence Co., Ltd., Gyeonggi-do (KR); VATECH EWOO Holdings Co., Ltd., Gyeonggi-do (KR)

(21) Appl. No.: 15/741,234

(22) PCT Filed: Jun. 30, 2016
(86) PCT No.: PCT/KR2016/007051

§ 371 Date: Dec. 29, 2017

(30) Foreign Application Priority Data
Jun. 30, 2015 (KR) 10-2015-0093294

(51) Int. Cl. A61B 6/02; A61B 6/00; G06T 7/00

(52) U.S. Cl. CPC A61B 6/022; A61B 6/5235; H04N 13/0207; G06T 2207/30068; G06T 7/0012; A61B 6/025; A61B 6/502; A61B 6/482

(57) ABSTRACT

The purpose of the present invention is to provide an image acquisition device and method capable of simultaneously acquiring a two-dimensional image and a three-dimensional image through a one-time photographing step. To this end, the present invention comprises: a control module for controlling a first photographing for acquiring a two-dimensional image and a second photographing for acquiring a three-dimensional image; an X-ray generation unit for irradiating an object, to be inspected, with X-rays in each of first and second doses at at least one position in accordance with the first photographing and at a plurality of second positions in accordance with the second photographing; an X-ray detector for receiving the X-rays by which the object to be inspected was irradiated, and acquiring data corresponding to the first and second photographings; and an image conversion unit for converting data of the first photographing into a two-dimensional image and converting data of the second photographing into a three-dimensional image, wherein the first dose is larger than the second dose, and the first position and the second positions are within a preset trajectory.

### TECHNICAL FIELD

The present invention relates to an image acquisition device and method for acquiring both of a two-dimensional image and a three-dimensional image through a single event of radiographic imaging.

### BACKGROUND ART

Generally, a mammography apparatus is an X-ray radiography apparatus for early detection of breast cancer. The apparatus projects a predetermined dose of X-rays toward the breast of a subject patient and detects an amount of X-rays that passes the object with a sensor of the apparatus, thereby acquiring a two-dimensional X-ray image (hereinafter, simply referred to as a two-dimensional image) or a three-dimensional X-ray image (hereinafter, simply referred to as a three-dimensional image).

Breast imaging methods for early detection or diagnosis of breast cancer are roughly categorized into full-field digital mammography (FFDM) mode, digital breast tomosynthesis (DBT) mode, and breast computed tomography (BCT) mode. The FFDM mode is a mode used to detect breast cancer early by acquiring a two-dimensional image. The DBT mode is a mode in which an X-ray source is rotated by a predetermined angle to improve a detection rate of a mass that cannot be easily identified in the FFDM mode. The BCT mode is a mode in which an X-ray source and a sensor are rotated by a predetermined angle or larger, thereby obtaining a three-dimensional image and accordingly providing precise position information of a lesion.

Meanwhile, there may be the case where it is required to acquire both of a two-dimensional image and a three-dimensional image for early detection and diagnosis of breast cancer, depending on conditions of patients. However, conventional mammography apparatuses for early detection of breast cancer are designed to obtain either a two-dimensional image or a three-dimensional image through a single event of radiographic imaging. Therefore, when it is required to acquire both of a two-dimensional image and a three-dimensional image, there is a problem that a patient must undergo a radiographic imaging process twice.

### DISCLOSURE

### Technical Problem

Accordingly, an objective of the present invention is to solve a problem with a conventional technology in which either a two-dimensional image or a three-dimensional image can be acquired through a single event of radiographic imaging and thus an object to be inspected needs to be exposed to X-rays at least two times when both of a two-dimensional image or a three-dimensional image are required.

Accordingly, another objective of the present invention is to provide an image acquisition device and method for simultaneously acquiring both of a two-dimensional image and a three-dimensional image through only a single event of radiographic imaging by projecting X-rays from each of a plurality imaging positions sequentially according to a first imaging order for acquiring a two-dimensional image and a second imaging order for acquiring a three-dimensional image, receiving an amount of X-rays passing through the object each time the X-rays are projected to obtain two-dimensional image data and three-dimensional image data, and reconstructing a two-dimensional image and a three-dimensional image from the acquired image data.

Objectives of the present invention may not be limited to the above-mentioned but other objectives and advantages of the present invention will be more clearly understood from the following detailed description taken in conjunction with embodiments disclosed below. In addition, it could be appreciated that the above and other objectives, advantages, and features of the present invention can be realized by means presented in the accompanying claims and combinations thereof.

### Technical Solution

In order to accomplish the above objective, the present invention provides an image acquisition device, including: a control module configured to control a first imaging operation for acquiring a two-dimensional image and a second imaging operation for acquiring a three-dimensional image; an X-ray generator configured to project a first dose of X-rays toward an object to be inspected from at least one first position preset for the first imaging operation and a second dose of X-rays toward the object from each of a plurality of second positions present for the second imaging operation; an X-ray detector configured to receive an amount of X-rays passing through the object and to acquire data corresponding to the first imaging operation and data corresponding to the second imaging operation; and an image converter configured to reconstruct a two-dimensional image from the data acquired through the first imaging operation and to reconstruct a three-dimensional image from the data acquired through the second imaging operation, wherein the first dose is larger than the second dose, and the first position and the second positions are on a predetermined trajectory.

In order to accomplish the above objective, the present invention provides an image acquisition method including: (a) projecting a first dose of X-rays toward an object to be inspected from a first position at which a first imaging operation is performed and a second dose of X-rays toward the object from each of a plurality of second positions at which a second imaging operation is performed; (b) receiving an amount of X-rays passing through the object and acquiring data corresponding to the first imaging operation and data corresponding to the second imaging operation; and (c) reconstructing a two-dimensional image and a three-dimensional image from the data acquired through the first imaging operation and the data acquired through the second imaging operation into a two-dimensional image and a three-dimensional image, wherein the first dose is larger than the second dose and the first position and the second positions are on a predetermined trajectory.

### Advantageous Effects

As described above, the present invention receives an amount of X-rays passing through an object after being projected from each of a plurality of imaging positions sequentially in a first imaging order for acquiring a two-dimensional image and a second imaging order for acquiring a three-dimensional image to acquire two-dimensional image and three-dimensional image data. Then, the present invention reconstructs a two-dimensional image and a three-dimensional image from the acquired data. In this way, the present invention can simultaneously acquire a two-dimensional image and a three-dimensional image through a single event of radiographic imaging.

In addition, since the present invention can simultaneously acquire a two-dimensional image and a three-dimensional image through a single event of radiographic imaging, it is possible to reduce patient inconvenience caused by two events of radiographic imaging.

### DESCRIPTION OF DRAWINGS

FIG. 1 is a diagram schematically illustrating an image acquisition device according to the present invention;

FIG. 2 is a block diagram illustrating an image acquisition device according to one embodiment of the present invention;

FIG. 3 is a diagram illustrating a radiographing that X-rays are projected from an imaging position at 0°;

FIG. 4 is a diagram illustrating a radiographing that X-rays are projected from each of imaging positions sequentially;

FIG. 5 is a diagram illustrating a radiographing that X-rays are sequentially projected toward an object at a part of each of imaging positions;

FIG. 6 is a diagram illustrating a radiographing that X-rays are sequentially projected toward an object at a part of each of imaging positions;

FIG. 7 is a flowchart illustrating an image acquisition method according to one embodiment of the present invention;

FIG. 8 is a flowchart illustrating details of Step S510 of the flowchart of FIG. 7; and

FIG. 9 is a flowchart illustrating details of Step S510 of the flowchart of FIG. 7.

### BEST MODE

The above and other objectives, features, and advantages of the present invention will be more clearly understood from the following detailed description taken in conjunction with the accompanying drawings. Thus, those skilled in the art would easily implement the technical spirit of the present invention. Further, when it is determined that the detailed description of the known art related to the present invention might obscure the gist of the present invention, the detailed description thereof will be omitted.

It will be understood that when an element is referred to as being “coupled” or “connected” to another element, it can be directly coupled or connected to the other element or electrically connected to the other element with an intervening element interposed therebetween. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof. As used herein throughout the entire specification, the singular forms “a”, “an”, and “the” are intended to include the plural forms as well, unless the context clearly indicates otherwise.

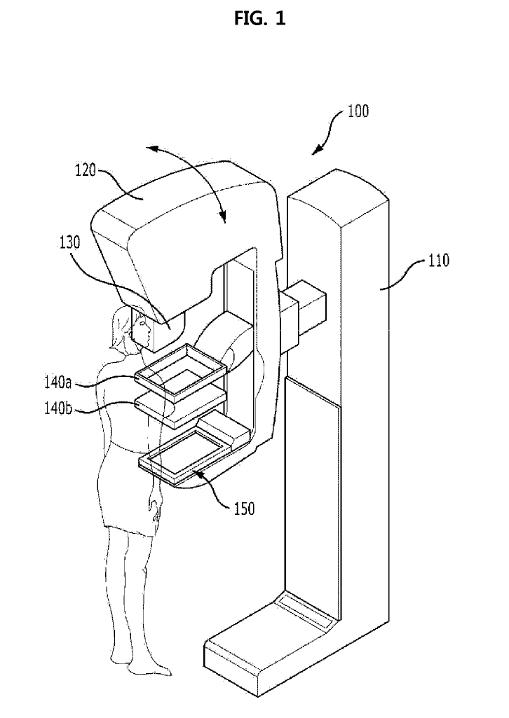

FIG. 1 is a diagram schematically illustrating an image acquisition device according to the present invention.

Referring to FIG. 1, an image acquisition device 100 includes a body 110, a gantry 120, an X-ray generator 130, a compression plate 140a, a supporting plate 140b, and an X-ray detector 150.

The body 110 includes an input device used for a radiologist to input commands for operating the image acquisition device 100, a display device displaying an image to allow the radiologist to see the image, a control module controlling the operation of the gantry 120 and the X-ray detector 150, and an image converter reconstructing a two-dimensional image and a three-dimensional image from the acquired data. The gantry 120 is mounted to one side surface of the body 110.

The X-ray generator 130 includes an X-ray source, projects X-rays to an object to be inspected, and is installed at an upper end portion of a front surface of the gantry 120.

The compression plate 140a is disposed between the X-ray generator 130 and the X-ray detector 150 and compresses a breast of a patient (object to be inspected) against the top surface of the supporting plate 140b such that the breast is in tight pressure contact with the top surface of the supporting plate 140b.

The supporting plate 140b is disposed between the compression plate 140a and the X-ray detector 150 and supports the breast from the underside.

The X-ray detector 150 detects X-rays that pass through the object after being projected toward the object by the X-ray generator 130, thereby acquiring data. The X-ray detector 150 is mounted on a detector rest which is integrated with the gantry 120. When the gantry 120 rotates, the X-ray detector 150 also rotates while directly facing the X-ray generator 130. Alternatively, a part of upper portion of the gantry 120 rotates so that the X-ray detector 150 is fixed and the X-ray generator 130 only rotates.

The X-ray detector 150 may include an X-ray sensor. The X-ray sensor is an area sensor in which a plurality of unit pixel sensors is two-dimensionally arranged, i.e., arranged in rows and columns to form a matrix of unit pixel sensors. The X-ray detector 150 receives X-rays projected by the X-ray generator 130 and having passed through a breast of a patient, thereby acquiring data of the amount of X-rays in connection with an interest region of the object to be inspected.

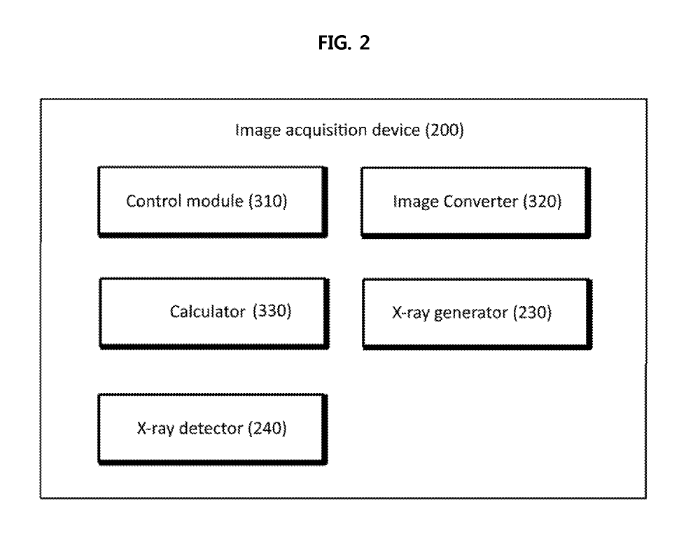

FIG. 2 is a block diagram illustrating an image acquisition device according to one embodiment of the present invention.

Referring to FIG. 2, according to one embodiment of the present invention, an image acquisition device 200 includes a control module 310, an image converter 320, an X-ray generator 230, and an X-ray detector 240.

The X-ray generator 230 includes an X-ray source to project X-rays toward an object to be inspected. The X-ray detector 240 includes an X-ray sensor to receive the X-rays projected by the X-ray generator 230 and thereby acquires data. The X-ray generator 230 projects X-rays toward the object from each of a plurality of imaging positions while rotating around the object. That is, the X-ray generator 230 projects X-rays in various directions. The X-ray generator 230 rotates within a rotation range of from −45° to +45° with respect to the object. That is, the imaging positions at each of which the X-ray generator 230 projects X-rays toward the object are within the rotation angle range of −45° to +45°. The rotation angle range may be more preferably an angular range of from −30° to +30°, and an interval between the adjacent imaging positions may be 1° to 5°, and it may be more preferably 2°.

Next, the control module 310 controls operation of the X-ray generator 230 and the X-ray detector 240, thereby controlling the imaging order in which an imaging operation for acquiring a two-dimensional image and an imaging operation for acquiring a three-dimensional image are performed.

The imaging order controlled by the control module 310 may be any one of the following orders: first imaging order in which imaging for acquiring a two-dimensional image is firstly performed and imaging for acquiring a three-dimensional image is performed next; second imaging order in which imaging for obtaining a part of a three-dimensional image is firstly performed to acquire a part of images for three-dimensional image, the X-ray generator 230 then moves to a position at which a two-dimensional image can be acquired and performs imaging for acquiring a two-dimensional image there, and imaging for acquiring the rest of the images for three-dimensional image is performed after the imaging for acquiring the two-dimensional image is completely performed; and third imaging order in which imaging for acquiring images for the three-dimensional image is firstly performed and imaging for acquiring a two-dimensional image is then performed. In regards to the first imaging order, the second imaging order, and the third imaging order, the imaging operation for acquiring a two-dimensional image is performed in a manner in which the X-ray generator 230 projects X-rays toward an object while being positioned at a 0° imaging position (hereinafter, referred to as a ‘central imaging position’) at which the X-ray generator 230 is disposed directly above the object and projects the X-rays are a direction perpendicular to the object. Meanwhile, the imaging operation for obtaining a three-dimensional image is performed in a manner in which the X-ray generator 230 projects X-rays toward the object sequentially from each imaging position other than the central imaging position by sequentially moving from one imaging position to another imaging position. The central imaging position may mean a cranial-caudal CC imaging position.

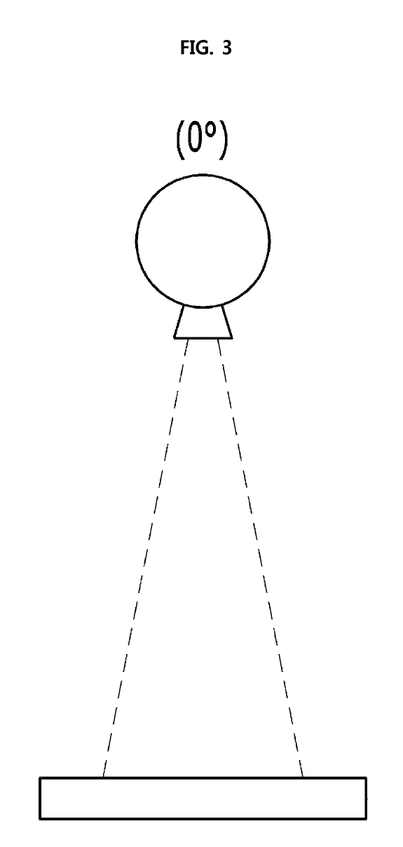

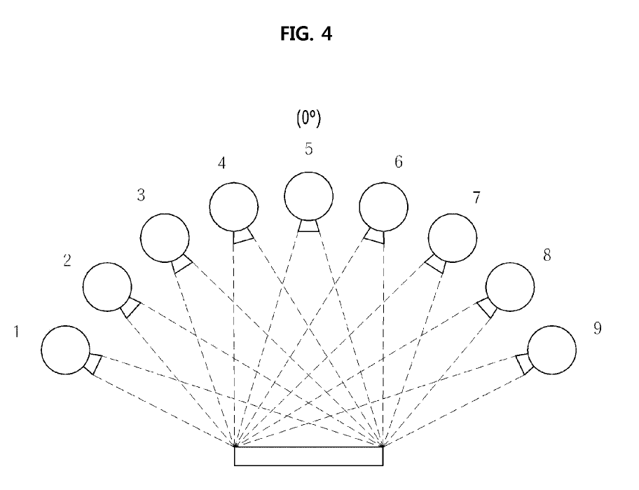

The first imaging order will be further described with reference to FIGS. 3 to 4.

The first imaging order is that the imaging for acquiring a two-dimensional image is performed firstly and then the other imaging for acquiring a three-dimensional image follows. More specifically, as illustrated in FIG. 3, the X-ray generator 230 firstly projects X-rays toward the object to acquire a two-dimensional image while being positioned at the central imaging position. Next, as illustrated in FIG. 4, the X-ray generator 230 projects X-rays toward the object from each of imaging positions 1 to 9 while moving from one imaging position to another imaging position. FIG. 4 illustrates only nine imaging positions. However, it is only an example, and the number of imaging positions may not be limited to nine.

Next, the third imaging order will be described below. As illustrated in FIG. 4, at each of the imaging positions ranging from position 1 to position 9, the X-ray generator 230 sequentially projects X-rays to acquire a three-dimensional image. Next, as illustrated in FIG. 3, at the central imaging position, the X-ray generator 230 projects X-rays to acquire a two-dimensional image.

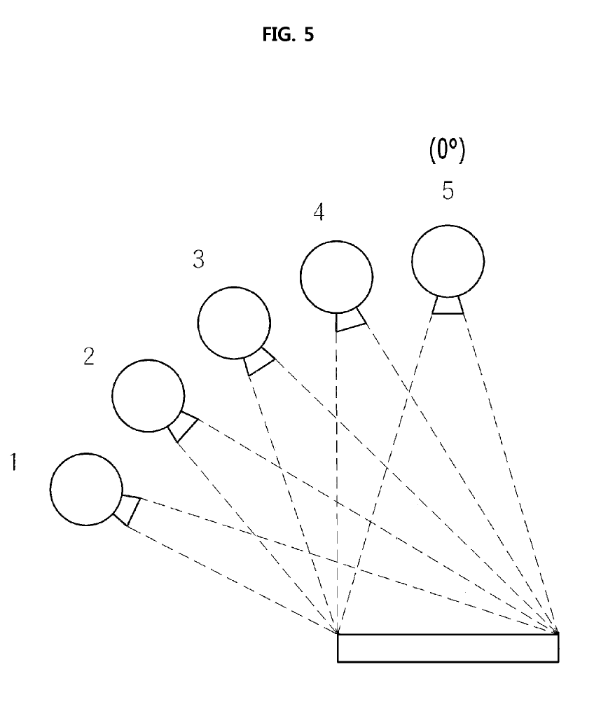

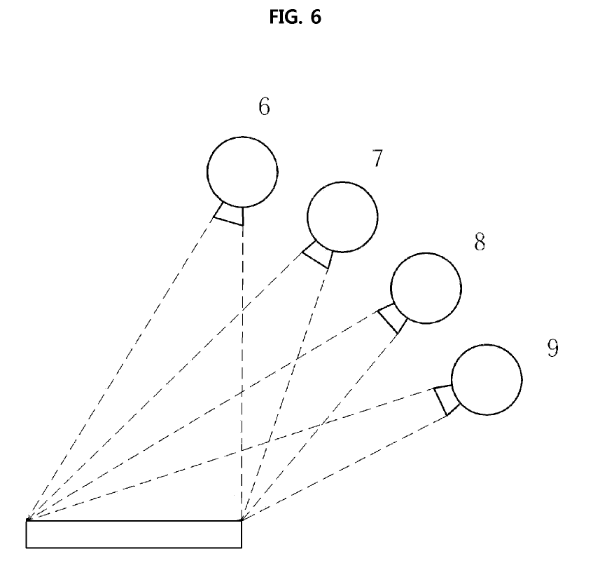

Next, the second imaging order will be described with reference to FIGS. 5 and 6.

The second imaging order means the following sequence: first, an imaging operation for acquiring a part of images for a three-dimensional image is performed, thereby acquiring a part of images for a three-dimensional image; next, the imaging position is transferred to the imaging position at which a two-dimensional image can be acquired, then performs an imaging operation for acquiring a two-dimensional image, thereby acquiring a two-dimensional image; and finally, an imaging operation for acquiring the rest of images for the three-dimensional image is performed. More specifically, as illustrated in FIG. 5, the X-ray generator 230 projects X-rays toward the object sequentially at each of imaging positions from one end imaging position 1 at one side to the central imaging position 5, thereby obtaining a part of images for a three-dimensional image. Next, the X-ray generator 230 performs an imaging operation for acquiring a two-dimensional image by projecting X-rays while being positioned at the central imaging position 5 (See FIG. 3). Next, as illustrated in FIG. 6, the X-ray generator 230 projects X-rays toward the object sequentially at each of imaging positions from a imaging position next to the central imaging position 5 to the other end imaging position at the other side 9, thereby performing an imaging operation for acquiring the rest of images for the three-dimensional image. Referring to the each of imaging positions 1 to 5 illustrated in FIG. 5 and the imaging positions 6 to 9 illustrated in FIG. 6, the X-ray projections for acquiring the three-dimensional image are performed at all of the imaging positions, as illustrated in FIG. 4. The total number of the imaging positions illustrated in FIGS. 5 and 6 is nine but it is exemplary. That is, the number may not be limited to nine.

Meanwhile, the control module 310 controls the projections of the X-ray generator 230 by a continuous shot manner in that the X-ray generator 230 rotates about an object and radiographs the object when in comes to an imaging position, or by a stop-and-shot manner in that the X-ray generator 230 completely stop and imaging at the each of the imaging position.

Alternatively, the X-ray generator 230 may include a plurality of X-ray sources disposed respectively at a plurality of imaging positions. In this case, the control module 310 controls the X-ray generator 230 such that the X-ray generator 230 does not rotate around the object but the X-ray sources sequentially project X-rays toward the object, according to the first imaging order or the second imaging order.

That is, in the case where a plurality of X-ray sources is disposed respectively at a plurality of imaging positions according to the above-described imaging principle, for example, in the case of an X-ray radiography apparatus disclosed in patent application No. PCT/KR2014/010618 by the current applicant, the control module 310 controls the X-ray generator 230 such that the X-ray generator 230 does not rotate around an object to be inspected but the X-ray sources disposed at the respective imaging positions sequentially project X-rays toward the object one after another according to the first imaging order or the second imaging order. In this case, the same result can be acquired. In this case, at least one of the X-ray sources may be a field emission type using a nanostructure field emission-type electron emitter.

Next, the image converter 320 reconstructs a two-dimensional image and a three-dimensional image from data acquired under the control of the control module 310. In this case, as described above, an X-ray sensor of the X-ray detector 240 may include a matrix-form pixel array in which unit pixels are arranged in rows and columns. Data obtained by the X-ray detector 240 by receiving X-rays is two-dimensionally arranged. Accordingly, for example, a two-dimensional image may be reconstructed from the obtained data obtained at a central imaging position, in complying with row and columns. Meanwhile, a three-dimensional image may be obtained by reconstructing the obtained data by projecting X-rays from each imaging position. This three-dimensional image may be a tomosynthesis image or a three-dimensional rendering image of volume data.

According to another embodiment of the present invention, an image acquisition device (mammography apparatus) for acquiring a two-dimensional image and a three-dimensional image may further include a calculator 330 that calculates doses of X-rays required for acquisition of a two-dimensional image and a three-dimensional image (See FIG. 2).

Specifically, before performing imaging according to the first imaging order or the second imaging order, the calculator 330 analyzes data obtained by detecting X-rays passing through an object to be inspected after projecting a low dose of X-rays and a normal dose of X-rays toward the object, and calculates an appropriate dose of X-rays required for acquisition of each of a two-dimensional image and a three-dimensional image on the basis of the data analysis result or information on the object such as height, age, etc. That is, the calculator 330 calculates an X-ray exposure voltage (kVp), a current (mAs), and an exposure time in accordance with the data analysis result or the information on the object. For example, when calculating an adequate exposure voltage, current, and time, the calculator 330 may first calculate the density of the object based on the acquired data, and determines a preset exposure voltage, current, and time in accordance with the calculated density.

In this case, the control module 310 performs control such that a dose of X-rays projected from each imaging position complies to the dose of X-rays calculated by the calculator 330.

For example, the control module 310 controls the X-ray generator such that a dose of X-rays projected to acquire a two-dimensional image is larger than a dose of X-rays projected to acquire a three-dimensional image. The dose of X-rays for acquisition of a two-dimensional image may be 8 to 12 mAs. For example, when the dose is determined to be about 10 mAs, the control module performs control such that the X-ray generator 230 projects X-rays in a dose of about 10 mAs from the central imaging position. The dose of X-rays for acquisition of a three-dimensional image may be 1 to 3 mAs for each imaging position. For example, when the dose is determined to be 2 mAs, the control module performs control such that the X-ray generator 230 projects X-rays in a dose of 2 mAs from each imaging position. The presented dose ranges are examples. Actual dose ranges can be adjusted depending on actual imaging conditions, the thickness of an object to be inspected, the position where the object is imaged, the number of times of imaging, etc. The dose of X-rays for a two-dimensional image may be determined to be larger than the dose of X-rays for a three-dimensional image while being under a dose limit for a single X-ray shot.

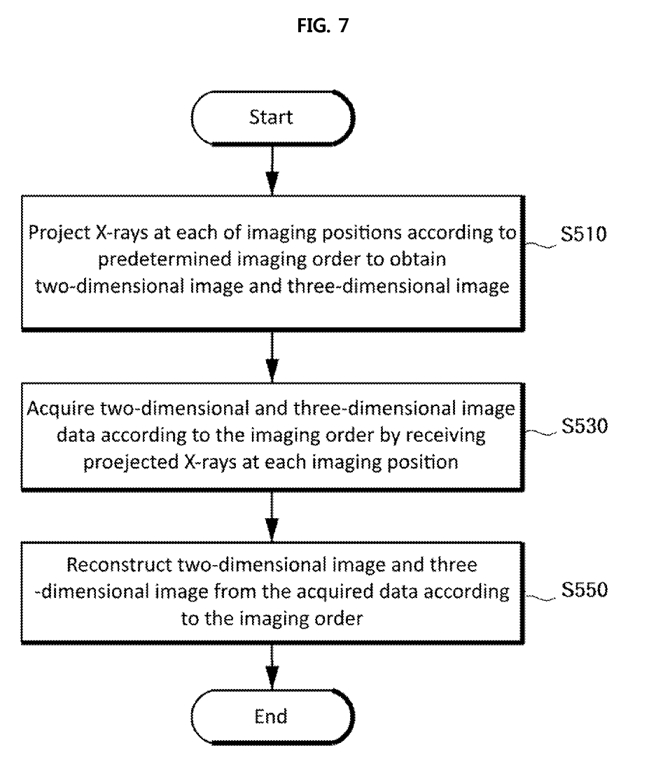

FIGS. 7 to 9 are flowcharts illustrating that an image acquisition method according to one embodiment of the present invention.

Referring to FIG. 7, X-rays are projected from each imaging position according to an imaging order for acquiring a two-dimensional image or an imaging order for acquiring a three-dimensional image (Step S510).

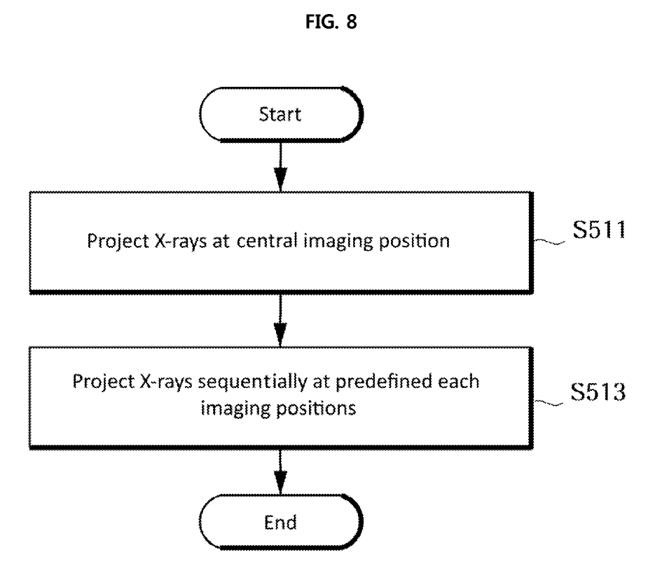

Referring to FIG. 8, Step S510 includes Step S511 of imaging at which X-rays are projected from a central imaging position to obtain a two-dimensional image and Step S513 of imaging at which X-rays are projected sequentially from each of the imaging positions to obtain a three-dimensional image.

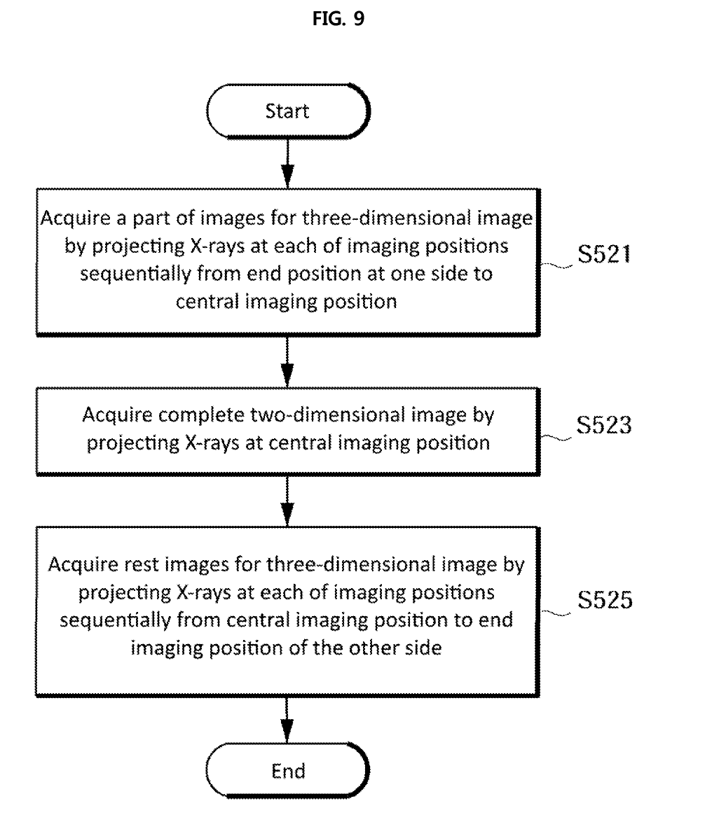

Alternatively, referring to FIG. 9, Step S510 may include: Step S521 of imaging at which X-rays is projected sequentially from each of the imaging positions one after another in order from one end imaging position at one side to the central imaging position, to obtain a part of images for a three-dimensional image; Step S523 of imaging at which X-rays are projected from the central imaging position to acquire a two-dimensional image; and Step S525 of imaging at which X-rays are projected sequentially at each of the imaging positions from the imaging position next the central imaging position to the other end imaging position at the other side, to obtain the rest of the images for the three-dimensional image.

Next, the X-rays that are projected from each imaging position and pass through the object are received to acquire two-dimensional image data and three-dimensional image data (Step S530).

Next, the data sequentially acquired according to the predetermined imaging order is reconstructed into a two-dimensional image and a three-dimensional image (Step S550).

Although the present invention has been described in conjunction with the preferred embodiment and the accompanying drawings, the present invention should not be construed as being limited to the embodiment. Those skilled in the art will appreciate that various modifications, additions, and substitutions are possible, without departing from the scope and spirit of the invention as disclosed in the accompanying claims.

Therefore, the scope of the present invention should not be constructed as being limited to the embodiment described above but rather should be defined only by the accompanying claims and their equivalents if appropriate.

1. An image acquisition device comprising:
a control module configured to control a first imaging operation for acquiring a two-dimensional image and a second imaging operation for acquiring a three-dimensional image;
an X-ray generator configured to project a first dose of X-rays toward an object to be inspected from at least one first position preset for the first imaging operation and a second dose of X-rays toward the object from each of a plurality of second positions present for the second imaging operation;
an X-ray detector configured to receive an amount of X-rays passing through the object and to acquire data corresponding to the first imaging operation and data corresponding to the second imaging operation; and
an image converter configured to reconstruct a two-dimensional image from the data acquired through the first imaging operation and to reconstruct a three-dimensional image from the data acquired through the second imaging operation,
wherein the first dose is larger than the second dose, and the first position and the second positions are on a predetermined trajectory.

2. The image acquisition device according to claim 1, wherein the first position is a central imaging position for the object, and the second positions are on both sides of the first position and spaced apart each other at predetermined intervals.

3. The image acquisition device according to claim 2, further comprising a calculator configured to calculate the first dose and the second dose in accordance with a state of the object.

4. The image acquisition device according to claim 1, wherein the X-ray generator includes at least one X-ray source, and the X-ray source projects X-rays toward the object from each of the first position and the second positions in a stop-end-shot manner or a continuous-shot manner.

5. The image acquisition device according to claim 1, wherein the X-ray generator includes a plurality of X-ray sources disposed at the first position and the second positions in one to one correspondence, and the X-ray sources project X-rays toward the object in turns.

6. An image acquisition method comprising:
(a) projecting a first dose of X-rays toward an object to be inspected from a first position at which a first imaging operation is performed and a second dose of X-rays toward the object from each of a plurality of second positions at which a second imaging operation is performed;
(b) receiving an amount of X-rays passing through the object and acquiring data corresponding to the first imaging operation and data corresponding to the second imaging operation; and
(c) reconstructing a two-dimensional image from the data acquired through the first imaging operation and a three-dimensional image from the data acquired through the second imaging operation,
wherein the first dose is larger than the second dose and the first position and the second positions are on a predetermined trajectory.

7. The image acquisition method according to claim 6, wherein the first position is a central imaging position for the object, and the second positions are on both sides of the first position and spaced apart each other at predetermined intervals.

8. The image acquisition method according to claim 6, wherein in the step (b), the X-rays are projected toward the object from the second position after the X-rays are projected toward the object from the first position.

9. The image acquisition method according to claim 6, wherein in the step (b), the X-rays are projected toward the object from the first position after the X-rays are projected toward the object from at least one of the second positions.

10. The image acquisition method according to claim 6, further comprising:
calculating the first dose of X-rays and the second dose of X-rays in accordance with a state of the object.

11. The image acquisition device according to claim 1, wherein the control module further configured to select one of the first imaging order and the second imaging order.

12. The image acquisition device according to claim 1, wherein the X-ray sources are a nanostructure field emission type.
